(19) United States
(12) Patent Application Publication
(10) Pub. No.: US 2025/0325471 A1
(43) Pub. Date: Oct. 23, 2025

(54) Preparation Method of Multidimensional Anti-Aging Nutrient

(71) Applicant: Andall Biosciences Inc., Torrance, CA (US)

(72) Inventor: Heju Li, Torrance, CA (US)

(21) Appl. No.: 18/741,990

(22) Filed: Jun. 13, 2024

(30) Foreign Application Priority Data
Apr. 23, 2024 (CN) 202410492757.0

(51) Int. Cl. A61K 8/9789; A61Q 19/08

(52) U.S. Cl. CPC A61K 8/9789; A61Q 19/08; A61K 2800/591; A61K 2800/84

(57) ABSTRACT

The present disclosure relates to the field of anti-aging technologies, and particularly to a preparation method of a multidimensional anti-aging nutrient. The multidimensional anti-aging nutrient includes anti-aging granules and a beauty nourishing oral liquid, wherein the anti-aging granules includes the raw materials: 10 g of Cistanche deserticola, 10 g of Morinda officinalis, 4 chrysanthemums, 10 g of Chinese wolfberry, ethanol, 10 g of lactose, 10 g of sucrose, 5 g of microcrystalline cellulose and 1 g of magnesium stearate; a process for preparing the anti-aging granules is as follows: step S1, soaking 10 g of Cistanche deserticola with yellow rice wine, then baking the soaked Cistanche deserticola, respectively smashing 10 g of Morinda officinalis without cores and 4 chrysanthemums without calyx to pass through a 20-mesh sieve, extracting the Cistanche deserticola with 60% ethanol, with a material-to-liquid ratio of 1:10, and then soaking for 30 min.

### CROSS REFERENCE TO RELATED APPLICATION

This application claims the benefit of and takes priority from Chinese Patent Application No. 202410492757.0 filed on Apr. 23, 2024, the contents of which are herein incorporated by reference.

### TECHNICAL FIELD

The present disclosure relates to the field of anti-aging technologies, and in particular to a preparation method of a multidimensional anti-aging nutrient.

### BACKGROUND

In the existing technology, people age as they grow older. Generally speaking, people can have wrinkles and gradual appearance of pigmentation, and subsequently the skin becomes dry, loose, rough and dull. At the same time, the skin's touch, pain and temperature senses weaken, and the surface reactivity weakens and is imbalanced, and regeneration and healing are easily stimulated. Accordingly, aging accompanies most of a person's life and has a negative impact on the work and life of beauty lovers. There is a great demand for anti-aging drugs worldwide, especially in wealthy countries. Delaying skin aging has become one of the research focuses of drugs. However, there is currently limited research on anti-aging drug raw materials, and the market's supply of anti-aging drug raw materials is not abundant enough, which limits the development of anti-aging drugs. Therefore, provided is a preparation method of a multidimensional anti-aging nutrient in order to solve the above problems.

### SUMMARY

The objective of the present disclosure is to provide a preparation method of a multidimensional anti-aging nutrient in order to solve the problems proposed in the above background.

The technical solution of the present disclosure is as follows: a preparation method of a multidimensional anti-aging nutrient, comprising a preparation process of anti-aging granules and a preparation method of a beauty nourishing oral liquid;
wherein, the anti-aging granules comprise the raw materials: 10 g of Cistanche deserticola, 10 g of Morinda officinalis, 4 chrysanthemums, 10 g of Chinese wolfberry, ethanol, 10 g of lactose, 10 g of sucrose, 5 g of microcrystalline cellulose and 1 g of magnesium stearate;
the preparation process of the anti-aging granules is as follows:
Step S1, soaking 10 g of Cistanche deserticola with yellow rice wine, then baking the soaked Cistanche deserticola, respectively smashing 10 g of Morinda officinalis without cores and 4 chrysanthemums without calyx to pass through a 20-mesh sieve, soaking Cistanche deserticola for 30 min and then performing reflux extraction on Cistanche deserticola with 60% ethanol in a material-to-liquid ratio of 1:10 for 1.5 h with twice extractions in total so as to obtain a Cistanche deserticola extracting solution, soaking Chinese wolfberry for 30 min and then performing reflux extraction on Chinese wolfberry with water in a material-to-liquid ratio of 1:12 for 1.5 h with three extractions in total so as to obtain a Chinese wolfberry extracting solution, extracting Morinda officinalis with 75% ethanol in a material-to-liquid ratio of 1:12 at the pH of 3 and then performing reflux extraction on Morinda officinalis with water in a material-to-liquid ratio of 1:12 for 1.5 h with twice extractions in total so as to obtain a Morinda officinalis extracting solution, performing reflux extraction on chrysanthemums with 60% ethanol in a material-to-liquid ratio of 1:5 at the pH of 5 for 1.5 h with twice extractions so as to obtain a chrysanthemum extracting solution, then evaporating the above extracting solutions by using a rotary evaporator to recover solvents at the temperature of below 70° C., and then concentrating the extracting solutions to obtain concentrated solutions;
Step S2, pre-freezing the concentrated solutions in an ultra-low temperature refrigerator for 3 h until the temperature reaches −50° C. or less, transferring the pre-frozen concentrated solutions to a freeze dryer which is pre-frozen for 30 min in advance to be subjected to freeze drying for 24 h, so as to obtain freeze-dried powders;
Step S3, weighing the freeze-dried powders, selecting 10 g of lactose, 10 g of sucrose and 5 g of microcrystalline cellulose in prescription amounts as fillers, passing the above materials through a 80-mesh sieve for later use, slowly adding a 60% ethanol solution into the screened materials to be uniformly mixed while continuously stirring so as to prepare a soft material having moderate hardness, passing the obtained soft material through a 14-mesh sieve for granulation, drying wet granules for 2 h in a drying oven at 60° C. and then adding 1 g of magnesium stearate while slowly adding the 60% ethanol solution to be uniformly mixed to prepare a soft material, passing the obtained soft material through the 14-mesh sieve for secondary granulation, then drying the prepared granules, subsequently passing the dried granules through a 10-mesh sieve, sizing and packaging to obtain the anti-aging granules;
the beauty nourishing oral liquid comprises the raw materials: 50 g of pearl powders, 3 pieces of turtle shells, 25 g of honeysuckle, 10 g of Poria cocos, 30 g of raw Rehmannia, 3.5 g of licorice, 300 g of sucrose, 1.5 g of sorbic acid, distilled water, 100 ml of concentrated ammonia test solution, 50% ethanol, and 36% concentrated hydrochloric acid;
the preparation method of the beauty nourishing oral liquid is as follows:
Step 4, taking 25 g of honeysuckle, 10 g of Poria cocos, 30 g of raw Rehmannia glutinosa and 3.5 g of licorice to be percolated according to a flow extract and a percolation method under the item of the extract, i.e., immersing the above materials for 24 h by using 50% ethanol as a solvent, then slowing percolating at the rate of 1-3 ml/min, collecting 850 ml of initial percolation solution to be stored in another vessel, continuing the percolation so that effective ingredients are completely percolated, collecting the further percolation solution, concentrating the further percolation solutions below 60° C. to form a viscous paste, adding the initial percolation solution into the viscous paste to be uniformly mixed, dropwise adding 100 ml of concentrated ammonia test solution so that the viscous paste is alkaline and has an ammonia odor, adjusting the concentration of the viscous paste by using 50% ethanol until 1 ml of viscous paste is equivalent to 1 g of raw medicine materials, standing, clarifying and filtering, so as to obtain the percolation solution;
Step S5, adding 25 g of pearl layer powders into distilled water, uniformly stirring, then adding a proper amount of 36% concentrated hydrochloric acid while stirring, until no bubbles are generated at which the dissolution is ended to obtain a pearl layer powder liquid;
Step S6, adding the pearl layer powder liquid into distilled water while continuously stirring, taking out supernatant, continuously washing the supernatant several times, measuring that the supernatant has no calcium ions by using an oxalic acid reagent, and discarding the supernatant obtained after washing to obtain a mother solution;
Step S7, performing suction filtration on the mother solution, collecting gelatinous substances, adding a proper amount of 36% concentrated hydrochloric acid, heating and refluxing for 24 h, cooling, taking out the mother solution, and heating the mother solution to remove a hydrogen chloride gas so as to obtain a concentrated solution;
Step S8, decocting 3 pieces of tortoise shells twice with water, with 2 h each time, combining decoction solutions, filtering and concentrating the combined decoction solution to obtain a tortoise shell solution; and
Step S9, adding the percolation solution, the concentrated solution and the tortoise shell solution into distilled water to be boiled, placing the above boiled solution overnight, filtering to obtain a filtrate for later use, preparing 300 g of sucrose into a simple syrup, dissolving 1.5 g of sorbic acid with 50% ethanol, adding the simple syrup and dissolved sorbic acid into the above filtrate so that the pH is 4.5-5.5, adding distilled water, uniformly stirring and subpackaging to obtain the beauty nourishing oral liquid.

Preferably, the Cistanche deserticola in step S1 is dried by using a fruit and vegetable drying machine.

Preferably, the rotary evaporator in step S1 adopts a RV8V rotary evaporator.

Preferably, the drying oven in step S3 adopts a drug drying oven and a precise drying oven.

Preferably, the vessel for decoction with water in step S8 adopts an enamel vessel or a stainless steel vessel, and the stainless steel adopts a 316 medical material.

Preferably, the vessel for preparing the percolation solution in step S4 adopts a cone-shaped percolation cylinder, and the length of the cylinder is 2-4 times the diameter of the cylinder.

Preferably, 350 ml of distilled water is added in step S5, 500 ml of distilled water is added in step S6, and 500 ml of distilled water is each added in step S9 for twice.

Preferably, the device for weighing freeze-dried powders in step S3 adopts a precise balance.

Through improvement, the present disclosure provides a preparation method of a multidimensional anti-aging nutrient here, which has the following improvements and advantages compared with the prior art:

1. In the present disclosure, the anti-aging granules are easy to store, correct in drug quantification, thorough in dehydration and less in volatile component loss, and facilitates the maintenance of the activity of heat-sensitive drugs, the nutrient of the anti-aging granules can nourishing kidney qi, the growth development and reproductive capacity of human are mainly determined by kidney yang and kidney essence, when kidney essence is sufficient, both men and women will become normal physiologically, and therefore supplementing kidney qi can improve overall aging.

2. In the present disclosure, the beauty nourishing oral liquid is a dark brown liquid with a sour and sweet taste and slightly bitter, and the various nutrients in the beauty nourishing oral liquid can provide nutrients such as protein, vitamins, minerals necessary for a body, can effectively remove acne, and have good effects on beauty nourishing, detoxification and detoxification.

3. In the present disclosure, first, an anti-aging facial mask is used to keep the skin moist, and then achieve the effect of tightening the skin, such the tightening effect helps to improve the skin's loose state, make the skin more elastic, and thus have a certain wrinkle removal effect. Second, the face is washed when the facial mask essence is almost absorbed, and then the beauty nourishing oral liquid is used to remove acne, and discharge toxins that accelerate aging in the body, the combination of them can nourish the beauty and resist the aging.

### BRIEF DESCRIPTION OF THE DRAWINGS

Next, the present disclosure will be further explained in combination with drawings and embodiments: influence of different dilution ratios on particle preparation.

FIG. 1 is a diagram showing the influence of different dilution ratios on granule preparation.

FIG. 2 is a diagram showing the influence of different magnesium stearates on granule preparation.

FIG. 3 is a diagram showing the influence of different ethanol concentrations on granule preparation.

FIG. 4 is a diagram showing the treatment standards for a beauty nourishing oral liquid.

### DETAILED DESCRIPTION OF THE EMBODIMENTS

Next, the present disclosure will be described in detail. The technical solution in the embodiments of the present disclosure will be described in clearly and completely, obviously, the described embodiments are only some embodiments of the present disclosure, but not all the embodiments. Based on the embodiments of the present disclosure, other embodiments obtained by persons of ordinary skill in the art without creative efforts are all included within the scope of protection of the present disclosure.

Through improvement, the present disclosure provides a preparation method of a multidimensional anti-aging nutrient here. The technical solution of the present disclosure is as follows:

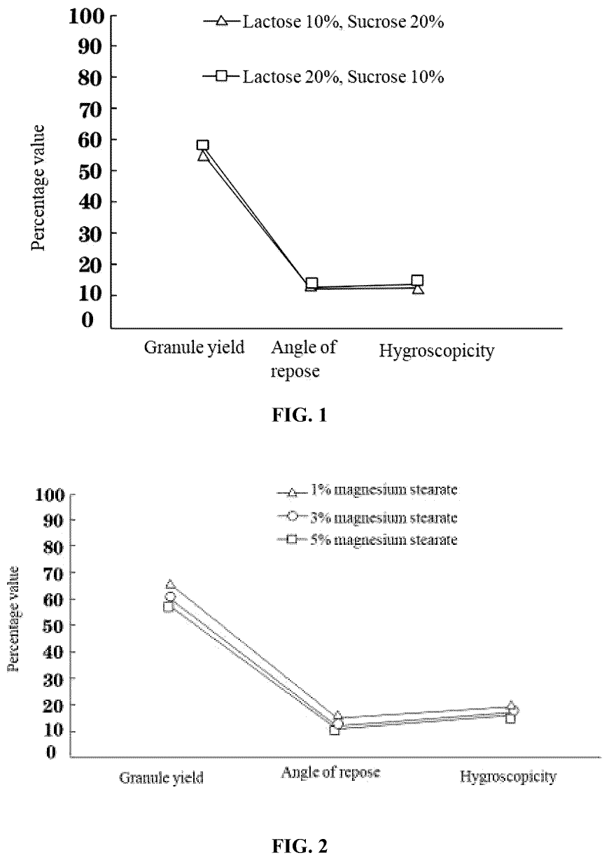

As shown in FIG. 1-FIG. 4, a preparation method of a multidimensional anti-aging nutrient, comprising a preparation process of anti-aging granules and a preparation method of a beauty nourishing oral liquid,
wherein, the anti-aging granules comprise the raw materials: 10 g of Cistanche deserticola, 10 g of Morinda officinalis, 4 chrysanthemums, 10 g of Chinese wolfberry, ethanol, 10 g of lactose, 10 g of sucrose, 5 g of microcrystalline cellulose and 1 g of magnesium stearate;
the preparation process of the anti-aging granules is as follows:
Step S1, soaking 10 g of Cistanche deserticola with yellow rice wine, then baking the soaked Cistanche deserticola, respectively smashing 10 g of Morinda officinalis without cores and 4 chrysanthemums without calyx to pass through a 20-mesh sieve, soaking Cistanche deserticola for 30 min and then performing reflux extraction on Cistanche deserticola with 60% ethanol in a material-to-liquid ratio of 1:10 for 2 h with twice extractions in total so as to obtain a Cistanche deserticola extracting solution, soaking 10 g of Chinese wolfberry for 30 min and then performing reflux extraction on Chinese wolfberry with water in a material-to-liquid ratio of 1:12 for 1.5 h with three extractions in total so as to obtain a Chinese wolfberry extracting solution, extracting Morinda officinalis with 75% ethanol in a material-to-liquid ratio of 1:12 at the pH of 3 and then performing reflux extraction on Morinda officinalis with water in a material-to-liquid ratio of 1:12 for 1.5 h with twice extractions in total so as to obtain a Morinda officinalis extracting solution, performing reflux extraction on chrysanthemums with 60% ethanol in a material-to-liquid ratio of 1:5 at the pH of 5 for 1.5 h with twice extractions so as to obtain a chrysanthemum extracting solution, then evaporating the above extracting solutions by using a rotary evaporator to recover solvents at the temperature of below 70° C., and then concentrating the extracting solutions to obtain concentrated solutions;

In this prescription, Chinese wolfberry and Cistanche deserticola are both rich in polysaccharide components, with high medicinal extractive viscosity and high medicinal extract extracting rate. The content of the extracts in the granules contain a large amount of extracts, making granules difficultly form and have poor fluidity. Therefore, a certain amount of magnesium stearate is selected as an anti-adhesive agent, as shown in FIG. 2;
Step S2, pre-freezing the concentrated solutions in an ultra-low temperature refrigerator for 3 h until the temperature reaches −50° C. or less, transferring the pre-frozen concentrated solutions to a freeze dryer which is pre-frozen for 30 min in advance to be subjected to freeze drying for 24 h, so as to obtain freeze-dried powders;
Step S3, weighing the freeze-dried powders, selecting 10 g of lactose, 10 g of sucrose and 5 g of microcrystalline cellulose in prescription amounts as fillers, passing the above materials through a 80-mesh sieve for later use, slowly adding a 60% ethanol solution into the screened materials to be uniformly mixed while continuously stirring so as to prepare a soft material having moderate hardness, passing the obtained soft material through a 14-mesh sieve for granulation, drying wet granules for 2 h in a drying oven at 60° C. and then adding 1 g of magnesium stearate while slowly adding the 60% ethanol solution to be uniformly mixed to prepare a soft material, passing the obtained soft material through the 14-mesh sieve for secondary granulation, then drying the prepared granules, subsequently passing the dried granules through a 10-mesh sieve, sizing and packaging to obtain the anti-aging granules;

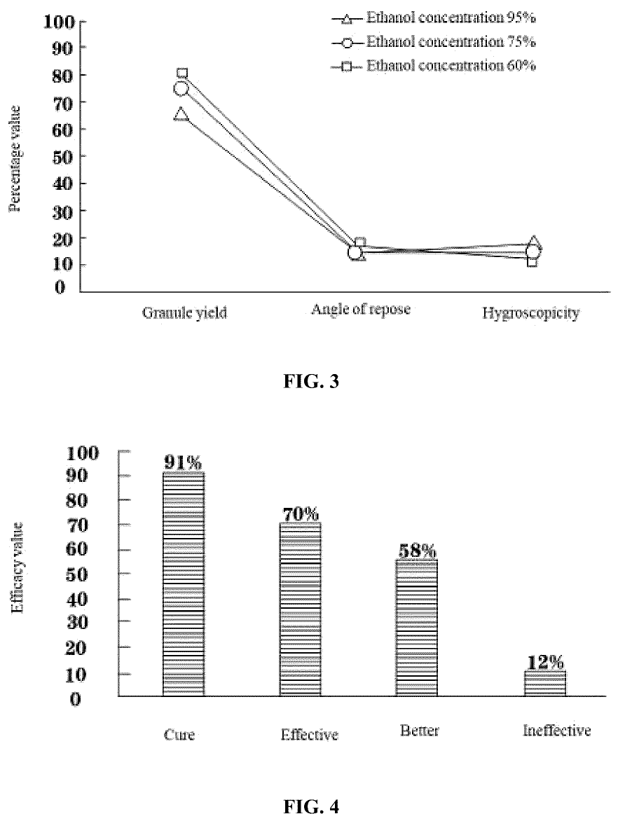

The medicinal extractive has a large viscosity and a certain hygroscopicity, and therefore is not suitable for serving as a wetting agent. Ethanol with an appropriate concentration is selected as the wetting agent, as shown in FIG. 3;

By using a funnel fixing method, 3 funnels are connected in series and fixed at a height (H) of 1 cm on horizontally placed coordinate paper, then drug powders made from different excipients are carefully poured into the top funnel along the funnel wall until the tip of the drug powder cone formed by the bottom funnel contacts the funnel mouth. The radius (R) of the cone bottom is measured by using coordinate paper and the angle (tg α=H/R) of rest is calculated. As shown in table 1, sucrose, dextrin, starch and lactose are commonly used diluents for anti-aging granular extractives which have a bitter and poor taste. Both sucrose and lactose have a sweet taste, they can also be used as flavor correction agents to make the taste of the granule better. Lactose has no hygroscopicity and stable properties, and is an excellent diluent, but has high cost. Sucrose is odorless, has a sweet taste and is stable in the air, but it is prone to moisture absorption and becoming damp, so as to affect the formability and hygroscopicity of granules, but it is inexpensive. Therefore, so they can be combined to save costs to a certain extent.
the beauty nourishing oral liquid comprises the raw materials: 50 g of pearl powders, 3 pieces of turtle shells, 25 g of honeysuckle, 10 g of Poria cocos, 30 g of raw Rehmannia, 3.5 g of licorice, 300 g of sucrose, 1.5 g of sorbic acid, distilled water, 100 ml of concentrated ammonia test solution, 50% ethanol, and 36% concentrated hydrochloric acid;
the preparation method of the beauty nourishing oral liquid is as follows:
Step 4, taking 25 g of honeysuckle, 10 g of Poria cocos, 30 g of raw Rehmannia glutinosa and 3.5 g of licorice to be percolated according to a flow extract and a percolation method under the item of the extract, i.e., immersing the above materials for 24 h by using 50% ethanol as a solvent, then slowing percolating at the rate of 1-3 ml/min, collecting 850 ml of initial percolation solution to be stored in another vessel, continuing the percolation so that effective ingredients are completely percolated, collecting the further percolation solution, concentrating the further percolation solutions below 60° C. to form a viscous paste, adding the initial percolation solution into the viscous paste to be uniformly mixed, dropwise adding 100 ml of concentrated ammonia test solution so that the viscous paste is alkaline and has an ammonia odor, adjusting the concentration of the viscous paste by using 50% ethanol until 1 ml of viscous paste is equivalent to 1 g of raw medicine materials, standing, clarifying and filtering, so as to obtain the percolation solution;
Step S5, adding 25 g of pearl layer powders into distilled water, uniformly stirring, then adding a proper amount of 36% concentrated hydrochloric acid while stirring, until no bubbles are generated at which the dissolution is ended to obtain a pearl layer powder liquid;
Step S6, adding the pearl layer powder liquid into distilled water while continuously stirring, taking out supernatant, continuously washing the supernatant several times, measuring that the supernatant has no calcium ions by using an oxalic acid reagent, and discarding the supernatant obtained after washing to obtain a mother solution;
Step S7, performing suction filtration on the mother solution, collecting gelatinous substances, adding a proper amount of 36% concentrated hydrochloric acid, heating and refluxing for 24 h, cooling, taking out the mother solution, and heating the mother solution to remove a hydrogen chloride gas so as to obtain a concentrated solution;
Step S8, decocting 3 pieces of tortoise shells twice with water, with 2 h each time, combining decoction solutions, filtering and concentrating the combined decoction solution to obtain a tortoise shell solution; and
Step S9, adding the percolation solution, the concentrated solution and the tortoise shell solution into distilled water to be boiled, placing the above boiled solution overnight, filtering to obtain a filtrate for later use, preparing 300 g of sucrose into a simple syrup, dissolving 1.5 g of sorbic acid with 50% ethanol, adding the simple syrup and dissolved sorbic acid into the above filtrate so that the pH is 4.5-5.5, adding distilled water, uniformly stirring and subpackaging to obtain the beauty nourishing oral liquid.

After the beauty nourishing oral liquid is administrated, cure: the acne disappears, the skin is smooth, and a total witnessed point is decreased by more than 90%; effective: the acne is decreased significantly, and a total witnessed point is decreased by 60%-90%; better: the acne is decreased, and a total witnessed point is decreased by 30%-60%; ineffective: the acne does not decrease or increase, but a total witnessed point is decreased by less than 20%, as shown in FIG. 4.

Further, the Cistanche deserticola in step S1 is dried by using a fruit and vegetable drying machine, the use of the fruit and vegetable drying machine can maintain the nutrients in food during the drying, such as vitamin and mineral substances, so that the nutrients in food cannot be excessively consumed or decomposed.

Further, the rotary evaporator in step S1 adopts a RV8V rotary evaporator. The use of the RV8V rotary evaporator helps to precisely control the distillation process, thereby obtaining more precise and reliable results.

Further, the drying oven in step S3 adopts a drug drying oven and a precise drying oven. The material is dried, sterilized and dried by using a drug drying oven, which can ensure the stability and purity of the material and improve the production efficiency of the material.

Further, the vessel for decoction with water in step S8 adopts an enamel vessel or a stainless steel vessel, and the stainless steel is a 316 medical stainless material. The enamel vessel is often used, because the surface of the enamel is smooth and does not easily adsorb bacteria and stains, it is convenient to clean.

Further, the vessel for preparing the percolation solution in step S4 adopts a cone-shaped percolation cylinder, and the length of the cylinder is 2-4 times the diameter of the cylinder. By using the cone-shaped percolation cylinder, the inclined cylinder wall can adapt to the volume change generated when the medicine is expanded. When the medicine has strong expansion, the use of the cone-shaped percolation cylinder can better handle the expansion to ensure the smooth progress of the percolation process.

Further, 350 ml of distilled water is added in step S5, 500 ml of distilled water is added in step S6, and 500 ml of distilled water is each added in step S9 for twice. The use of the distilled water can supplement the electrolyte required by the body and maintain the electrolyte balance of the body.

Further, the device for weighing freeze-dried powders in step S3 adopts a precise balance. The precise balance adopts an advanced electronic sensor and measurement technology, so as to achieve the precise measurement of the minor quality changes to obtain more correct and reliable measurement results.

The above descriptions make those skilled in the art achieve or use the present disclosure. Various modifications to these embodiments are obvious to those skilled in the art. The general principle defined herein can be implemented in other embodiments without departing the spirit or scope of the present disclosure. Therefore, the present disclosure will not be limited to these embodiments disclosed herein, but meet the widest scope consistent to the principle and novel features disclosed herein.

What is claimed is:

1. A preparation method of a multidimensional anti-aging nutrient, comprising a preparation process of anti-aging granules and a preparation method of a beauty nourishing oral liquid,
wherein, the anti-aging granules comprise the raw materials: 10 g of Cistanche deserticola, 10 g of Morinda officinalis, 4 chrysanthemums, 10 g of Chinese wolfberry, ethanol, 10 g of lactose, 10 g of sucrose, 5 g of microcrystalline cellulose and 1 g of magnesium stearate;
the preparation process of the anti-aging granules is as follows:
step S1, soaking 10 g of Cistanche deserticola with yellow rice wine, then baking the soaked Cistanche deserticola, respectively smashing 10 g of Morinda officinalis without cores and 4 chrysanthemums without calyx to pass through a 20-mesh sieve, soaking Cistanche deserticola for 30 min and then performing reflux extraction on Cistanche deserticola with 60% ethanol in a material-to-liquid ratio of 1:10 for 1.5 h with twice extractions in total so as to obtain a Cistanche deserticola extracting solution, soaking Chinese wolfberry for 30 min and then performing reflux extraction on Chinese wolfberry with water in a material-to-liquid ratio of 1:12 for 1.5 h with three extractions in total so as to obtain 10 g of Chinese wolfberry extracting solution, extracting Morinda officinalis with 75% ethanol in a material-to-liquid ratio of 1:12 at the pH of 3 and then performing reflux extraction on Morinda officinalis with water in a material-to-liquid ratio of 1:12 for 1.5 h with twice extractions in total so as to obtain a Morinda officinalis extracting solution, performing reflux extraction on chrysanthemums with 60% ethanol in a material-to-liquid ratio of 1:5 at the pH of 5 for 1.5 h with twice extractions so as to obtain a chrysanthemum extracting solution, then evaporating the above extracting solutions by using a rotary evaporator to recover solvents at the temperature of below 70° C., and then concentrating the extracting solutions to obtain concentrated solutions;
Step S2, pre-freezing the concentrated solutions in an ultra-low temperature refrigerator for 3 h until the temperature reaches −50° C. or less, transferring the pre-frozen concentrated solutions to a freeze dryer which is pre-frozen for 30 min in advance to be subjected to freeze drying for 24 h, so as to obtain freeze-dried powders;
Step S3, weighing the freeze-dried powders, selecting 10 g of lactose, 10 g of sucrose and 5 g of microcrystalline cellulose in prescription amounts as fillers, passing the above materials through a 80-mesh sieve for later use, slowly adding a 60% ethanol solution into the screened materials to be uniformly mixed while continuously stirring so as to prepare a soft material having moderate hardness, passing the obtained soft material through a 14-mesh sieve for granulation, drying wet granules for 2 h in a drying oven at 60° C. and then adding 1 g of magnesium stearate while slowly adding the 60% ethanol solution to be uniformly mixed to prepare a soft material, passing the obtained soft material through the 14-mesh sieve for secondary granulation, then drying the prepared granules, subsequently passing the dried granules through a 10-mesh sieve, sizing and packaging to obtain the anti-aging granules;
the beauty nourishing oral liquid comprises the raw materials: 50 g of pearl powders, 3 pieces of turtle shells, 25 g of honeysuckle, 10 g of Poria cocos, 30 g of raw Rehmannia, 3.5 g of licorice, 300 g of sucrose, 1.5 g of sorbic acid, distilled water, 100 ml of concentrated ammonia test solution, 50% ethanol, and 36% concentrated hydrochloric acid;
the preparation method of the beauty nourishing oral liquid is as follows:
Step 4, taking 25 g of honeysuckle, 10 g of Poria cocos, 30 g of raw Rehmannia glutinosa and 3.5 g of licorice to be percolated according to a flow extract and a percolation method under the item of the extract, i.e., immersing the above materials for 24 h by using 50% ethanol as a solvent, then slowing percolating at the rate of 1-3 ml/min, collecting 850 ml of initial percolation solution to be stored in another vessel, continuing the percolation so that effective ingredients are completely percolated, collecting the further percolation solution, concentrating the further percolation solutions below 60° C. to form a viscous paste, adding the initial percolation solution into the viscous paste to be uniformly mixed, dropwise adding 100 ml of concentrated ammonia test solution so that the viscous paste is alkaline and has an ammonia odor, adjusting the concentration of the viscous paste by using 50% ethanol until 1 ml of viscous paste is equivalent to 1 g of raw medicine materials, standing, clarifying and filtering, so as to obtain the percolation solution;
Step S5, adding 25 g of pearl layer powders into distilled water, uniformly stirring, then adding a proper amount of 36% concentrated hydrochloric acid while stirring, until no bubbles are generated at which the dissolution is ended to obtain a pearl layer powder liquid;
Step S6, adding the pearl layer powder liquid into distilled water while continuously stirring, taking out supernatant, continuously washing the supernatant several times, measuring that the supernatant has no calcium ions by using an oxalic acid reagent, and discarding the supernatant obtained after washing to obtain a mother solution;
Step S7, performing suction filtration on the mother solution, collecting gelatinous substances, adding a proper amount of 36% concentrated hydrochloric acid, heating and refluxing for 24 h, cooling, taking out the mother solution, and heating the mother solution to remove a hydrogen chloride gas so as to obtain a concentrated solution;
Step S8, decocting 3 pieces of tortoise shells twice with water, with 2 h each time, combining decoction solutions, filtering and concentrating the combined decoction solution to obtain a tortoise shell solution; and
Step S9, adding the percolation solution, the concentrated solution and the tortoise shell solution into distilled water to be boiled, placing the above boiled solution overnight, filtering to obtain a filtrate for later use, preparing 300 g of sucrose into a simple syrup, dissolving 1.5 g of sorbic acid with 50% ethanol, adding the simple syrup and dissolved sorbic acid into the above filtrate so that the pH is 4.5-5.5, adding distilled water, uniformly stirring and subpackaging to obtain the beauty nourishing oral liquid.

2. The preparation method of the multidimensional anti-aging nutrient according to claim 1, wherein the Cistanche deserticola in step S1 is dried by using a fruit and vegetable drying machine.

3. The preparation method of the multidimensional anti-aging nutrient according to claim 1, wherein the rotary evaporator in step S1 adopts a RV8V rotary evaporator.

4. The preparation method of the multidimensional anti-aging nutrient according to claim 1, wherein the drying oven in step S3 adopts a drug drying oven and a precise drying oven.

5. The preparation method of the multidimensional anti-aging nutrient according to claim 1, wherein the vessel for decoction with water in step S8 adopts an enamel vessel or a stainless steel vessel, and the stainless steel adopts a 316 medical material.

6. The preparation method of the multidimensional anti-aging nutrient according to claim 1, wherein the vessel for preparing the percolation solution in step S4 adopts a cone-shaped percolation cylinder, and the length of the cylinder is 2-4 times the diameter of the cylinder.

7. The preparation method of the multidimensional anti-aging nutrient according to claim 1, wherein 350 ml of distilled water is added in step S5, 500 ml of distilled water is added in step S6, and 500 ml of distilled water is each added in step S9 for twice.

8. The preparation method of the multidimensional anti-aging nutrient according to claim 1, wherein the device for weighing freeze-dried powders in step S3 adopts a precise balance.
